(19) United States
(12) Patent Application Publication
(10) Pub. No.: US 2026/0083528 A1
(43) Pub. Date: Mar. 26, 2026

(54) DEVICE FOR SUPPORTING AN OPERATOR

(71) Applicant: HELLSTERN MEDICAL GMBH, Wannweil (DE)

(72) Inventors: Sabrina HELLSTERN, Wannweil (DE); Claudia SODHA, Gechingen (DE); Alexander STROBEL, Haigerloch-Stetten (DE); Harald RAGER, Bisingen-Wessingen (DE)

(21) Appl. No.: 19/111,649

(22) PCT Filed: Sep. 11, 2023
(86) PCT No.: PCT/EP2023/074888

§ 371 Date: Mar. 13, 2025

(30) Foreign Application Priority Data
Sep. 15, 2022 (DE) 10 2022 123 626.9

(51) Int. Cl. A61B 90/60; A47C 3/20

(52) U.S. Cl. CPC A61B 90/60; A47C 3/20

(57) ABSTRACT

The device according to the present disclosure serves to support an operator (surgeon). The device comprises a mobile platform a base column, extending from the platform with a seat an upper body holding device and at least one actuating unit The actuating unit is arranged in an area between a seat surface of the seat and the platform in such a way that the actuating unit can be reached by the surgeon with his foot, so that a surgeon sitting on the seat of the device can easily reach the actuating unit with his foot, in particular with his heel or sole, from any position.

### CROSS REFERENCE TO RELATED APPLICATIONS

The present application is a National Stage of PCT Application No. PCT/EP2023/074888 filed on Sep. 11, 2023, which claims priority to German Patent Application No. 10 2022 123 626.9 filed on Sep. 15, 2022, the contents each of which are incorporated herein by reference thereto

### TECHNICAL FIELD

The present disclosure relates to a device for supporting an operator or surgeon respectively, particularly configured to support an operator's body during a medical operation, such as a surgical procedure.

### BACKGROUND

Support systems for operators or surgeons respectively, such as operating or surgical chairs, are generally known and serve to support the operator, who usually stands at the operating table, in such a way that he can perform the often long-lasting medical operations in forced postures or medical operations with special demands on the operator's fine motor skills, without fatigue or in a fatigue-free manner, and ideally perform them in a reliable and high-quality.

DE 10 2020 103 861 B3 discloses an operating chair or surgical chair respectively that serves to support an operator. The operator can sit on the surgical chair during an operation. This surgical chair comprises a seat supported by a height-adjustable column, wherein the column stands on a joint device that defines two tilt axes for the column and which is assigned to a locking device, in order to lock manually adjusted tilt positions, so that the column retains a desired tilt position. The column extends from a walkable platform on the upper side, of which several foot-operated control switches are arranged, with which the column can be locked and released. The platform also serves for the operator or surgeon to place his feet on the platform during the operation.

In an operating room, there is usually only limited space available for the operating table and the additional equipment, such as an operating chair. This limits the dimensions of the operating chair and thus also the platform of the operating chair. In particular, the size of the area of the platform where the operator or surgeon respectively can place his feet during the operation is limited. The operating chair should also be drivable as close as possible to the surgical area to minimize the distance between the operator sitting on the operating chair and the operating site and to offer the operator (surgeon) a comfortable operating position or surgical position respectively as possible. The control switches can be configured as foot pedals, for example. The foot-operated control switches for operating the surgical chair are arranged in this limited area of the platform, which, however, can cause the operator (surgeon) to trip over the control switches when entering the platform and/or accidentally and/or incorrectly operate the control switches during the operation.

### BRIEF SUMMARY

Based on this, it is the object of the present disclosure to provide an improved device for supporting an operator (surgeon) that reduces the risk of tripping or the risk of accidental misoperation of the device by the operator (surgeon).

Disclosed is a device for supporting an operator comprising a platform from which an adjustable base column with a seat attached thereto protrudes, and an upper body holding device with at least one traction means, at an end of which a harness worn by the operator can be coupled and decoupled, wherein at least one actuating unit for controlling the base column and/or the upper body holding device is arranged in an area between a seat surface of the seat and the platform such that the actuating unit can be reached by the operator with his foot.

The device according to the present disclosure comprises a platform from which extends a height-adjustable base column with an attached seat for the operator (surgeon). In addition, the device comprises an upper body holding device with a traction means at the end of which a harness worn by the operator can be coupled and uncoupled. The device also has at least one actuating unit for controlling the base column and/or the upper-body support device, which is arranged in an area between a seat surface of the seat and the platform, so that the actuating unit is reachable by the foot of the operator (surgeon), preferably with the heel or the sole of the foot. The actuating unit is particularly ergonomic as the operator (surgeon) reaches the actuating unit with his foot without overextending the foot or leg.

As a result, the actuating unit for controlling the base column and/or the upper-body support device is arranged in a space-saving way on the platform and in particular it is not arranged in the front area of the platform, where the operator (surgeon) can place his feet during the medical operation (surgery). The platform can thus be exclusively configured for placing the operator's feet, thereby reducing the risk of tripping or misoperation by the operator.

A key feature of the device according to the present disclosure in particular concerns the actuating unit being arranged below the seat, thereby enabling particularly ergonomic and intuitive operation of the actuating unit, and the actuating unit is space-saving on the platform. The operator (surgeon) can kick backwards with his foot in a relatively gross motorized manner in order to reach the actuation unit, for example with his heel or the sole of his foot, without having to concentrate on the actuation process.

In particular, the actuating unit is arranged at least substantially perpendicular to the platform, whereby the actuating unit can be provided with a large actuating surface which the operator can use to control the device. The operator can thus reach the actuation unit with the foot without having to concentrate on the actuation process. In addition, the cleaning and/or disinfection of the device required before or after an operation can be made easier, as the platform can have a simpler design, for example by the top of the platform being designed as a flat surface.

The actuating unit may have several, preferably at least two, separately operable actuating elements that the operator can operate with his foot. An actuating direction of the actuating elements of the actuating unit is preferably aligned parallel to each other and/or to the surface of the platform.

The platform may have a trapezoidal or rectangular outline. In particular, it may also have a T-shaped outline so that it's narrow end can move forward under an operating table and reduce the risk of collision with a column supporting the operating table. The device preferably has an electrically driven drive device arranged below the platform. The device can preferably be moved along at least two directions of travel using the drive device.

The adjustable base column can extend (in a basic position) at least substantially perpendicular to the platform. For example, the adjustable base column can be height-adjustable and/or laterally pivotable and/or pivotable forwards and backwards. The traction means of the upper body holding device may be, for example, a belt, a strap or a (wire) rope.

The upper body holding device may have an abutment for the traction means, for example with an electrically driven winch on which the traction means can be wound and unwound. By winding or unwinding the traction means on the winch, the length of the traction means can thus be changed by the upper body holding device. If the end of the traction means is coupled to a harness worn by the operator, the angle of inclination at which the operator's upper body is held can be adjusted by changing the length of the traction means. The operator's harness may be an upper body harness for the operator, in which a coupling connection is arranged on the operator's back when he wears the harness.

In particular, the actuating unit is arranged in an area which extends at least substantially between the seat and the platform. The area in which the actuating unit is arranged is in particular truncated pyramid-shaped with a top surface defined at the height of the seat and a base surface defined at the height of the platform, the centers of which are preferably arranged perpendicular to one another. The top surface and the base surface of the truncated pyramid can be formed, for example, as a polygon, rectangle, square or circle. If the seat is height-adjustable, the top surface of the area is preferably defined at the height of the lowest possible height setting of the seat.

The top surface of the area can be determined by a unit seating area, which is defined as an area within which the dimensions of the hip of an average operator (surgeon) projected onto the unit seating area lie in any case. For example, the unit seating area can be defined as a square, with a width and a length of at least substantially 0.4 m. If the unit seating area is defined as a circle, the diameter of the unit seating area can be at least substantially 0.4 m.

The base area of the area may be determined by a unit standing area, which is defined as an area within which the dimensions of the shoulders of an average operator (surgeon) projected onto the unit standing area lie. For example, the unit standing area may have a width of at least substantially 0.6 m and a length of at least substantially 0.4 m if the unit standing area is defined as a rectangle. If the unit standing area is defined as a circle, the diameter of the unit standing area can be at least substantially 0.6 m. In particular, between the base of the truncated pyramid and the front of the platform there is a tread surface for the operator (surgeon), on which the operator (surgeon) can at least partially place his feet.

Preferably, the actuating unit is configured to switch an operating mode of the upper body holding device and/or the base column upon actuation. For example, the base column and/or the upper body holding device may initially be in an operating mode in which the settings of the base column and/or the upper body holding device are locked. When the actuating unit is actuated, it may switch the operating mode of the base column and/or the upper body holding device to an operating mode in which the base column or the length of the traction means of the upper body holding device is movable, i.e. not locked, whereby the device can be adjusted by the operator (surgeon). When actuated again, the actuating unit can, for example, set the operating mode of the base column and/or the upper body holding device to a locked state again.

Preferably, the platform has a front side facing an operating area during an operation, wherein the actuating unit is arranged facing the front side. This makes it easier for the operator (surgeon) to reach the actuating unit with his heel or the sole of his foot.

At least one tread surface for the feet of the operator is provided on an upper side of the platform, in particular between the base column and the front side of the platform. The tread surface may, for example, be at least partially flat. The surgeon can place or set down his feet on the tread surface during the operation.

It is preferred that the actuating unit has at least one actuating element with an actuating surface where the actuating element is movable between a first position and a second position and is resiliently (spring-loaded) in the first position. The actuating surface may be at least partially flat and/or at least partially concave. The actuating direction in which the actuating elements are movable from the first position to the second position is preferably at least substantially horizontal. In particular, the actuating unit defines an actuating pivot axis about which the actuating element is pivotable to actuate the actuating unit, the actuating pivot axis extending at least substantially parallel to the surface of the platform. The actuating pivot axis is preferably arranged below the surface of the platform, wherein the actuating unit is triggered independently of the height at the actuating element at which the operator touches the actuating element with his foot when stepping backwards. The first position of the actuating element is preferably arranged closer to the tread surface than the second position of the actuating element.

Preferably, the actuating unit is configured to send a switchover signal to a control device of the base column and/or the upper body holding device when the actuating element is in the second position.

In particular, the actuating surface of the actuating element is arranged at a predetermined angle relative to the tread surface. The predetermined angle between the actuating surface and the tread surface may, for example, be between 100° and 45°, particularly between 95° and 50°, preferably between 90°and 60°, especially preferably at least substantially 90°. The vertical arrangement of the actuating surface relative to the tread surface can make it easier for the operator (surgeon) to reach the actuating unit with his foot, for example with his heel or the sole of his foot.

It is preferred the adjustable base column is at least laterally pivotable about a pivot axis. Preferably, the base column is configured to be height-adjustable.

Preferably, the actuating surface of the actuating unit is at least rectangular. This is the simplest possible design of the actuating surface. A height-to-width ratio of the actuating surface is in particular greater than 2:1, preferably greater than 3:1, particularly preferably greater than 4:1. In particular, the area of the actuating surface is 0.06 m² or more, in particular 0.07 m² or more. The actuating surface is thus configured to be as large as possible, so that it can be hit as easily as possible by the operator's foot (surgeon's foot).

In an alternative embodiment, the actuating unit is attached to the base column or integrated into the base column. The base column and thus also the actuating unit are arranged at least substantially below the seat surface of the seat but nevertheless within reach of the operator's foot, preferably with the heel or the sole of the foot. The platform of the device can thus be exclusively configured as a standing surface of the operator's feet. The fixed arrangement of the actuating unit on the adjustable base column and the integration of the actuating unit in the adjustable base column may also be advantageous, as the actuating unit is always fixed in relation to the adjustable base column. When the base column is adjusted relative to the platform, for example by pivoting the base column sideways and/or pivoting the base column forwards or backwards, the position and/or orientation of the actuating unit also changes with the base column. This can additionally improve the operability of the actuation unit for the operator (surgeon).

In this embodiment, the height above the platform in which the actuating unit is arranged is particularly one third of the height of the truncated pyramid or less, preferably one fourth of the height of the truncated pyramid or less, particularly preferably one fifth of the height of the truncated pyramid or less.

The actuating surface of the actuating unit is particularly arranged at a predetermined distance from the platform, the distance being preferably greater than 0.05 m, especially greater than 0.10 m, particularly preferably greater than 0.15 m. As a result, the actuating unit can be operated particularly easily with the foot, enabling the operator to trigger the actuating unit and at the same time maintain the same weight, for example also in strongly inclined positions.

In a specific embodiment, the actuating unit particularly has at least two actuating elements, which are preferably arranged in the heel area of the right and left legs of the operator. The at least two actuating elements of the actuating unit are designed so that they can be actuated independently of one another.

### BRIEF DESCRIPTION OF THE DRAWINGS

Further details of advantages, further embodiments or details of the present disclosure result from the drawings, the description and the claims. The Figures show:

FIG. 1 a perspective view of a first embodiment of the device for supporting the operator according to the present disclosure.

FIG. 2 a perspective view of a second embodiment of the device for supporting the operator according to the present disclosure.

FIG. 3 a detailed view of the actuating unit according to the first embodiment.

FIG. 4 a detailed view of the actuating unit according to the second embodiment and

FIG. 5 a detailed view of the actuating unit according to a third embodiment.

### DETAILED DESCRIPTION

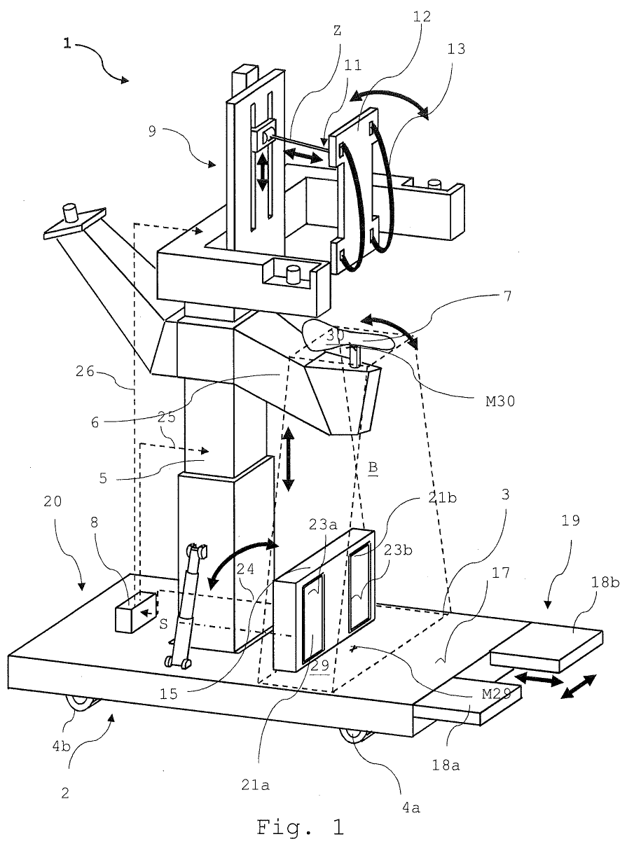

FIG. 1 shows a first embodiment of the device 1 for supporting an operator (surgeon). The device 1 features a walkable platform 3, under which a drive device 2 is arranged. The drive device 2 can have at least three, for example, electrically driven rollers. At least one of the three electrically driven rollers can be rotatable about a vertical axis. In FIG. 1, the drive device 2 has four electrically driven rollers, with only two of the four rollers 4a, 4b, shown in FIG. 1. In the drive device 2 illustrated in FIG. 1, at least two of the rollers are configured to be rotatable about a vertical axis. The device 1 can thus be driven into a desired position.

The device 1 also comprises a base column 5, which extends at least substantially vertically from the platform 3. The base column 5 shown in FIG. 1 is height adjustable and laterally pivotable. The lateral pivoting takes place about a lateral pivot axis S, which is defined in a plane formed by the platform 3.

The device 1 from FIG. 1 also comprises an upper body holding device 9. In the example shown in FIG. 1, the upper body holding device 9 is attached to the base column 5. Alternatively, the upper body holding device 9 can also be arranged separately on the platform 3, for example behind the base column 5. The upper body holding device 9 comprises a traction means Z at whose end 11, a harness 13 worn by the operator can be coupled and uncoupled. In the example shown in FIG. 1, the harness 13 also has a back plate 12, on which suspension points for the harness 13 are attached. The back plate 12 of the harness 13 can also have a coupling connection, wherein the end 11 of the traction means Z comprises a connection, so that the end 11 of the traction means Z, can be detachably connected to the harness 13. The coupling connection of the harness 13 and the connection at the end 11 of the traction means Z are preferably configured to be complementary to one another.

The upper body holding device 9 shown in FIG. 1 forms an abutment for the traction means Z, on which the upper body of the operator (surgeon) can be coupled. For this purpose, the upper body holding device 9 comprises, for example, an electrically driven winch with which the traction means Z, can be wound and unwound. The upper body holding device 9 is preferably arranged behind the operator located in the device 1, so that the operator can lean his upper body forward, with the upper body of the operator being held in a certain position by the traction means. By controlling the length of the traction means, the inclination angle of the operator can first be set at which he receives holding support.

A seat 7 is also attached to the base column 5, on which the operator (surgeon) can sit. In the example shown in FIG. 1, a longitudinal beam 6 is attached to an upper end of the base column 5, which extends—starting from the base column 5—towards the front side 19 of the platform 3. The longitudinal beam can be arranged at least substantially horizontally. At the front end of the longitudinal beam 6, the seat 7 is arranged, on which the operator can sit.

The device 1 has an actuating unit 15, arranged in an area B between the seat 7 and the platform 3, which is arranged perpendicularly relative to the surface of the platform 3. The area B extends below the seat 7 from the seat towards the platform 3, wherein the area B always extends behind the tread surface 17. The area B, in which the actuating unit 15 can be arranged, is a truncated pyramid with a square top surface 30 and a rectangular base surface 29, the centers M29, M30 of which are positioned vertically one above the other. The top surface is defined by a unit seat surface and the base surface is defined by a unit standing surface.

In the example shown in FIG. 1, the actuating unit 15 has two actuating elements 21a and 21b. Each of the actuating elements 21a and 21b has an actuating surface 23a and 23b, respectively. The actuating surfaces 23a, 23b are large. The length of the actuating surfaces 23a, 23b can, for example, correspond to at least a half, a third or a quarter of the height of the area B. An operator sitting on the seat 7 can thus reach the actuating surfaces 23a and 23b with his foot.

The platform 3 has a front side 19 and a rear side 20, the front side 19 being the side that faces the operating area during the operation. The base column 5 is arranged between the front side 19 and the rear side 20 of the platform 3. The base column 5 can, for example, be arranged at least substantially centrally in the platform. Between the front side 19 and the base column 5, a tread surface 17 is formed on the upper side of the platform 3, which the operator (surgeon) can step on when entering the platform and can place his feet on after sitting down on the seat. The tread surface 17 provides the operator with a certain amount of support, which is required if the operator wants to change the height of the base column or pivot the base column 5 sideways, for example.

In the example shown in FIG. 1, the actuating element 21a is assigned to the operator's right leg and the actuating element 21b is assigned to the operator's left leg. Additionally, further actuating elements can be provided in the actuating unit 15.

The actuating unit 15 is communicatively connected to a control device 8. A signal line 24 connects the actuating unit 15 to the control device 8. When one of the actuating surfaces 23a, 23b is activated, the actuating unit 15 sends a switching signal to the control device 8. Control device 8 is also communicatively connected to the adjustable base column 5 and the upper body holding device 9. The connection between the control device 8 and the base column 5 and the upper body holding device 9 canbe individually configured, for example, via the signal lines 25 and 26. Alternatively, communication can also take place via a bus system.

In FIG. 1 two additional support surfaces 18a and 18b are additionally attached to the front side 19 of the platform 3. The additional support surfaces 18a and 18b can be used as support surfaces for any foot pedals of instruments. This means that the tread surface 17 remains free of foot pedals, so that there is no risk of the operator (surgeon) tripping.

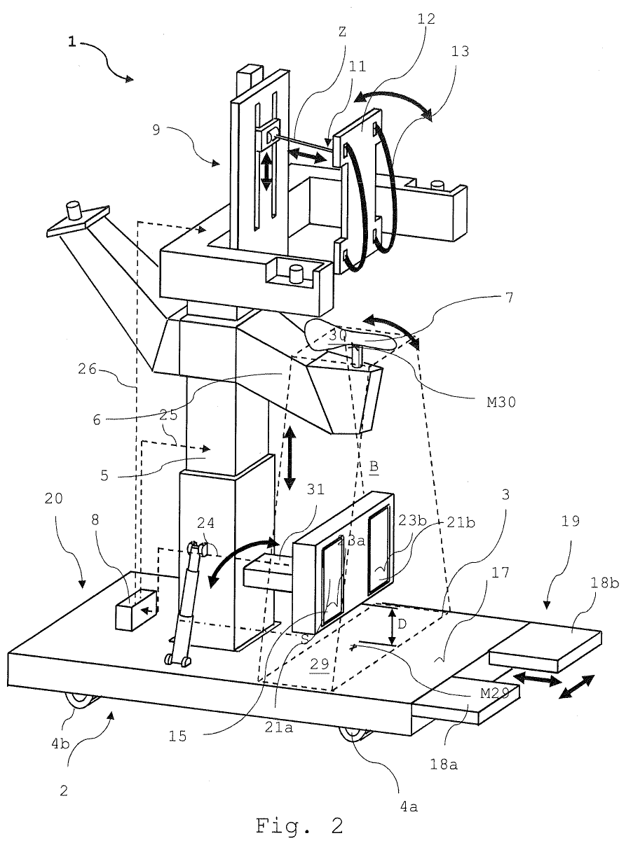

FIG. 2 shows a second embodiment of the device 1. The second embodiment differs from the first embodiment in that the actuating unit 15 for controlling the base column 5 and the upper body holding device 9 is attached to the base column 5 or integrated into it. In the device 1 shown in FIG. 2, the actuating unit 15 is attached to the base column 5. The actuating unit 15 is arranged at a predetermined distance D from the platform 3. Alternatively, the actuating unit 15 can also be integrated into the base column 5. The actuating unit 15 is arranged in an area B between the seat 7 and the platform 3 and is attached to the base column 5 using a fastening element 31. The area B extends below the seat 7 from the seat towards the platform 3, wherein the area B always extends behind the tread surface 17. The area B in which the actuating unit 15 can be arranged is a truncated pyramid with a square top surface 30 and a rectangular base surface 29, the centers of which are positioned vertically one above the other. The height above the platform 3 in which the actuating unit 15 is positioned is approximately a quarter of the height of the truncated pyramid in the example shown in FIG. 1.

By attaching the actuating unit 15 to the base column 5 or integrating the actuating unit 15 in the base column 5, the actuating unit 15 always remains in a certain fixed relation to the operator's (surgeon's) own position, even under large swivel angles or greatly variable height settings of the base column 5. The operator can thus more easily find the actuating unit with his foot without being forced to look at where the actuating unit 15 is currently located.

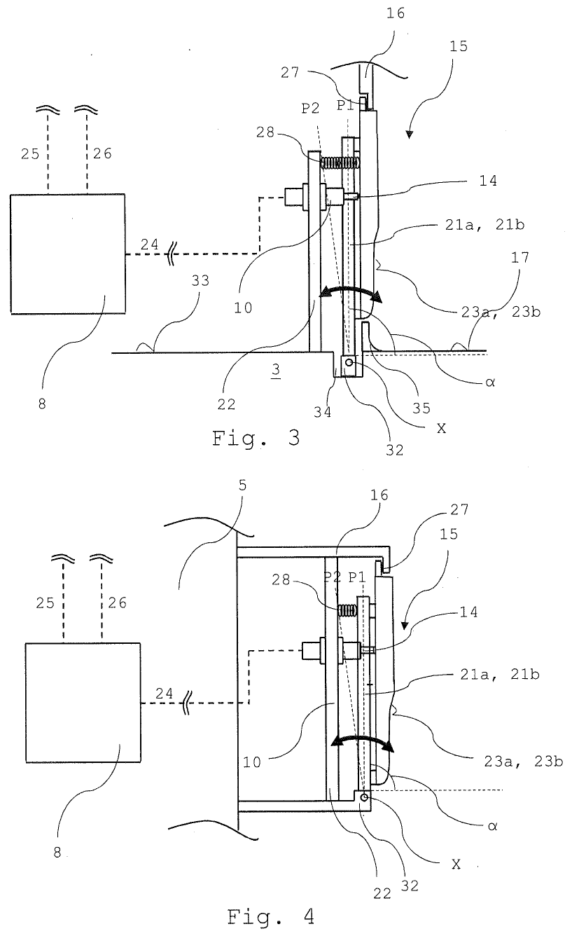

FIG. 3 shows an example of the actuating unit 15 in cross-section. In this example, the actuating unit 15 is attached to the platform 3 and is oriented at least substantially perpendicular to the platform 3. On a side of the actuating unit 15 facing the front side 19 of the platform 3 there is an actuating element 21a, 21b, which is held spring-loaded in a first position P1.

The actuating element 21a, 21b can be moved from the first position P1 to a second position P2 against the spring force of a spring element 28. The spring element 28 can define the pressure with which the actuating element 21a, 21b is to be actuated. The spring element 28 can be, for example, configured as a non-linear compressing spring. The first position P1 of the actuating element 21a, 21b is defined by a stop surface 27 provided in a housing 16 of the actuating element 15. The actuating element 21a, 21b has an actuating surface 23a, 23b against which the operator can press during actuation. The actuating element 21a, 21b is provided with at least one hinge 32 which defines an actuating axis X, about which the actuating element 21a, 21b is pivotable. In the example shown, the actuating axis X is arranged at a lower end of the actuating element 21a, 21b. The actuating axis X extends at least substantially parallel to the surface 33 of the platform 3. However, the hinge 32 can also be arranged at a right, left or upper end of the actuating element 21a, 21b. In the example shown in FIG. 3, the hinge 32, which defines the actuating axis X, is housed in a recess 34 below the surface 33 of the platform 3 and attached to the platform 3. This can prevent the operator from accidentally hitting the hinge 32 with his foot instead of the actuation surface 23a, 23b, which would not trigger the actuation unit 15. In FIG. 3, a protective element 35 is also attached to the surface 33 of the platform 3 below the actuation surface 23a, 23b. The protective element 35 separates the actuating unit 15 from the tread surface 17.

In the example shown in FIG. 3, the actuation surface 23a, 23b is approximately orthogonally to the tread surface 17. The angle α between the tread surface 17 and the actuating surface 23a, 23b is approximately 90° when the actuation element 21a, 21b is in the first position P1.

A switch element 10 is arranged within the actuating unit 15. The switch element 10 is attached to the platform 3 with a positioning element 22. The switch element 10 has an actuating pin 14 which rests against the back side of the actuating element 21a, 21b. When the actuating element 21a, 21b is pushed into the second position P2, the actuating pin 14 is pressed into the switch element 10. The switch element 10 is connected to the control device 8 via the signal line 24. When the actuating element 21a, 21b is in the second position P2, the switch element 10 sends a switching signal to the control device 8. The control device 8 is communicatively connected to the base column 5 and the upper body holding device 9 via the signal lines 25, 26.

Unlike as illustrated in FIG. 3, the spring element 28 can also be arranged within the switch element 10.

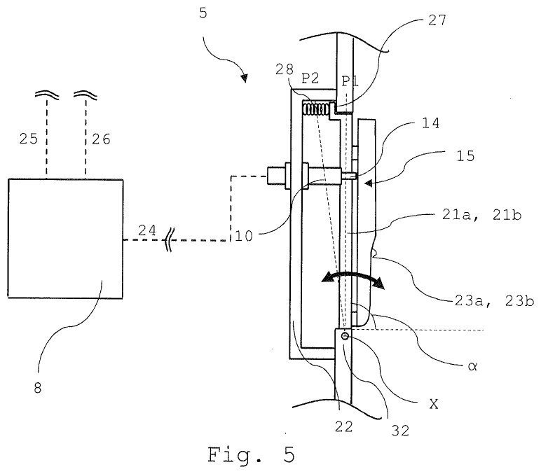

FIG. 4 shows a further example of the actuating unit 15 in cross-section. The example shown in FIG. 4 corresponds to the example from FIG. 3, considering the reference numerals, but the actuating unit 15 is attached to the base column 15.

FIG. 5 shows a further example of the actuating unit 15 in cross-section. In this example, the actuating unit 15 is integrated into the base column 5. This means that the actuating unit 15 does not have a separate housing. The actuating unit 15 is rather housed in the housing 29 of the base column 5.

The features shown in FIGS. 3 to 5 can be combined.

The device according to the present disclosure serves to support a surgeon. The device 1 has a mobile platform 3, a base column 5 protruding from the platform 3 with a seat 7, an upper body holding device 9, and at least one actuation unit 15. The actuation unit 15 is arranged in an area B between a seat surface of the seat 7 and the platform 3 in such a way that the actuation unit 15 can be reached by the surgeon with his foot, so that a surgeon sitting on the seat 7 of the device 1 can easily reach the actuation unit with his foot, in particular with his heel or sole of his foot, from any position.

### REFERENCE SIGNS

1 device
2 drive device
3 platform
4a, b, c electrically driven rollers
5 base column
6 longitudinal beam
7 seat
8 control device
9 upper body holding device
10 switch element
11 end of the traction means
12 back plate of the harness
13 harness
14 actuating pin
15 actuating element
16 housing of the actuating element
17 tread surface
18a, b support surface
19 front side of the platform
20 rear side of the platform
21a, b actuating element
22 positioning element
23a, b actuating surface
24 signal line from the actuating unit to the control device
25 signal line from the control device to the base column
26 signal line from the control device to the upper body holding device
27 stop of the actuating element
28 spring element
29 base surface
30 top surface
31 fastening element
32 hinge
33 surface of the platform
34 recess in the platform
B area
D distance between the actuating unit and the plat form
H main axis of the base column
P1 first position
P2 second position
S pivot axis
X actuating swivel axis
Z traction means
α angle between the actuating surface and the tread surface

1. A device for supporting an operator comprising a platform from which an adjustable base column with a seat attached thereto protrudes, and an upper body holding device with at least one traction means at an end of which a harness worn by the operator can be coupled and decoupled,
wherein at least one actuating unit for controlling the base column and/or the upper body holding device is arranged in an area between a seat surface of the seat and the platform such that the actuating unit can be reached by the operator with his foot.

2. The device according to claim 1, wherein the area is truncated pyramid-shaped with a top surface defined at a height of the seat and a base surface defined at a height of the platform a center of the top surface being vertically arranged above a center of the base surface.

3. The device according to claim 1, wherein the actuating unit is substantially perpendicular to the platform.

4. The device according to any of the preceding claim 1, wherein the platform has at least one front side which faces an operating area during an operation, the actuating unit being arranged facing the front side

5. The device according to claim 4, wherein at least one tread surface for a foot of the operator is provided on the upper side of the platform between the actuating unit and the front side of the platform.

6. The device according to claim 1, wherein the actuating unit comprises at least one actuating element with an actuating surface that is movable between a first position and a second position and a spring held in the first position.

7. The device according to claim 6, wherein the actuating unit defines an actuating pivot axis (X) about which the at least one actuating element is pivotable, wherein the actuating pivot axis extends at least substantially parallel to the a surface of the platform.

8. The device according to claim 7, wherein the actuating pivot axis is arranged below the surface of the platform.

9. The device according to claim 5, wherein the actuating unit comprises at least one actuating element with an actuating surface that is movable between a first position and a second position and a spring held in the first position and the first position is arranged closer to the tread surface than the second position.

10. The device according to claim 1, wherein the actuating unit is configured to switch an operating mode of the base column and/or the upper body holding device when actuated.

11. The device according to claim 6, wherein the actuating unit is configured to send a switching signal to a control device of the base column and/or the upper body holding device when the at least one actuating element is in the second position.

12. The device according to claim 5, characterized in that wherein the actuating unit comprises at least one actuating element with an actuating surface that is movable between a first position and a second position and a spring held in the first position, the actuating surface of the at least one actuating element is arranged at a predetermined angle relative to the at least one tread surface

13. The device according to claim 12, wherein the predetermined angle between the actuating surface and the at least one tread surface is between 100° and 45°, and 50°.

14. The device according to claim 1, wherein the base column is configured to be laterally pivotable about a pivot axis.

15. The device according to claim 1, wherein the actuating unit is attached to the base column or integrated into a base column.

16. The device according to claim 6, wherein the actuating surface is substantially rectangular, wherein a height-to-width ratio of the actuating surface is greater than 2:1.

17. The device according to to claim 6, wherein the actuating surface has an area of 0.06 m² or more.

18. The device as in claim 13, wherein the predetermined angle is between 95° and 50° or between 90° and 60°.

19. The device as in claim 16, wherein the height-to-width ratio of the actuating surface is greater than 3:1 or greater than 4:1.

20. The device as in claim 17, wherein the actuating surface has an area of 0.07 m² or more.
